(19) United States
(12) Patent Application Publication
(10) Pub. No.: US 2013/0288385 A1
(43) Pub. Date: Oct. 31, 2013

(54) MOLECULARLY IMPRINTED TETRAETHOXYSILANE POLYMER AND METHODS OF USING THE SAME TO DETECT SECOSTEROIDS

(71) Applicants: Wade A. Jensen, Clearfield, UT (US); Joseph J. BelBruno, Hanover, NH (US); Yuan Liu, West Lebanon, NH (US)

(72) Inventors: Wade A. Jensen, Clearfield, UT (US); Joseph J. BelBruno, Hanover, NH (US); Yuan Liu, West Lebanon, NH (US)

(73) Assignee: TRUSTEES OF DARTMOUTH COLLEGE, Hanover, NH (US)

(21) Appl. No.: 13/825,631

(22) PCT Filed: Sep. 23, 2011
(86) PCT No.: PCT/US11/52891

§ 371 Date: Mar. 22, 2013

Related U.S. Application Data
(60) Provisional application No. 61/386,218, filed on Sep. 24, 2010.

(51) Int. Cl. G01N 33/543

(52) U.S. Cl. CPC G01N 33/54386
USPC 436/501; 422/69

(57) ABSTRACT

A molecular imprinted tetraethoxysilane polymer device for detecting secosteroids, or metabolites thereof, and a method for using the same are provided.

This patent application claims the benefit of priority from U.S. Provisional Application Ser. No. 61/386,218, filed Sep. 24, 2010, the content of which is incorporated herein by reference in its entirety.

### BACKGROUND OF THE INVENTION

Molecular imprinting is a technique that allows for the production of molecule specific receptors that are analogous to biological receptor binding sites without the cost or environmental sensitivity of the natural systems (Shea (1994) Trends Polym. Sci. 2:166; Wulff (1995) Angew. Chem. Int. Ed. 34:1812; Mosbach & Ramstrom (1996) Biotechnology 14:163; BelBruno (2009) Micro and Nanosystems 1:163). Molecularly imprinted polymers (MIPs) may be based on either covalent or non-covalent binding between the host polymer and the target or template molecule. Various MIP-based devices have been suggested for use in the detection of surface-binding molecules, inorganic compounds, organic compounds, polymers, biological molecules, nanoparticles, viruses, and biological arrays (WO 2008/063204 and US 2009/0115605). Numerous host polymers have been used in the preparation of MIPs, including 4-vinyl pyridine, nylon, and methacrylate. In addition, tetraethoxysilane (TEOS) has been suggested for use in producing MIPs that recognize sulfonamides (Lee, et al. (2010) J. Polymer Res. 17:737-744) or dye molecules (U.S. Pat. No. 7,923,082).

Cholecalciferol, or Vitamin D₃, is a secosteroid hormone that plays an important role in the regulation of calcium absorption, bone mineralization, and bone mineral density. Vitamin D₃ deficiencies can cause rickets and osteomalacia (Laird, et al. (2010) Nutrients 2:693-724). In addition, it has been suggested that Vitamin D₃ deficiencies are associated with cardiovascular disease, symptoms of depression, and cognitive deficits (Barnard, et al. (2010) Am. J. Geriatric Parmacother. 8:1-31). These are common deficiencies for third-world and aged persons. Vitamin D₃ is naturally synthesized by the upper epidermus upon exposure to UVB radiation (290-315 cm⁻¹), with full production reached after 10-15 minutes of sun exposure. This depends, however; on skin pigmentation, geographical location, season, and age. Locations of greater latitude, receive less UVB, and sufficient Vitamin D₃ synthesis only occurs in the summer (Laird, et al. (2010) supra).

### SUMMARY OF THE INVENTION

The present invention features a device and kit for detecting a secosteroid, or metabolite thereof. The device of the invention is composed of a support, and a molecular imprinted tetraethoxysilane polymer film having sites of selective recognition for a secosteroid, or metabolite thereof. In one embodiment, the film is produced by phase inversion-spin coating on the support. A method for using device to detect a secosteroid, or metabolite thereof, in a test sample is also provided.

### DETAILED DESCRIPTION OF THE INVENTION

A MIP produced with TEOS has now been developed for use in the adsorption of Vitamin D₃. Time trials showed that one gram of Vitamin D₃ per one liter of methanol mixed with equal volume of water had a higher rate of adsorption than the other solvents used (chloroform and ethanol).

Accordingly, the present invention features a device for detecting secosteroids such as Vitamin D₃ and methods for using the same. The device according to the present invention includes at least one molecular imprinted film having sites of selective recognition of one or more secosteroids. Preferably, the molecular imprinted film is produced using TEOS. Molecular Imprinted Polymers (MIPs) are obtained by polymerization of functional monomers, with or without cross-linker, in the presence of template molecules. The functional monomers organize specifically around the template molecule, then after polymerization, their functional groups form a highly cross-linked polymer structure. After polymerization, the template molecule is extracted from the polymer. The resulting polymer then has sites that are complementary in size, form or location with template molecule and capable of recognizing the template molecule with very great specificity. The polymer can then selectively adsorb the template molecule when it is put into contact with the same.

In the present invention, the template molecule is a secosteroid or metabolite thereof. As is known in the art, secosteroids are similar in structure to steroids with the exception that two of the B-ring carbon atoms (C9 and 10) of the typical four steroid rings are not joined. Secosteroids of use in the instant device and methods include Vitamin D₃ (also known as cholecalciferol) and Vitamin D₂ (ergocalciferol) and metabolites thereof including 25-hydroxyvitamin D₃ (also known as calciferol or calcidiol), 25-hydroxyvitamin D₂, 1α,25-dihydroxyvitamin D₃ (also known as calcitriol); 1α-hydroxyvitamin D₂, 1α-hydroxyvitamin D₃, or 24-hydroxyvitamin D₂. Given that the instant TEOS-based MIP could readily detect Vitamin D₃ in solution, it is contemplated that the instant MIP can be prepared with other steroids or other metabolic intermediates as templates. Such steroids or metabolic intermediates include, e.g., 11-deoxycortisol, 21-deoxycortisol, 17-alpha-hydroxyprogesterone, aldosterone, 4-androstene-3,17-dione, corticosterone, deoxycorticosterone, cortisol, dehydroepiandrosterone, dehydroepiandrosterone sulfate, estradiol, estriol, estrone, progesterone, 5-alpha-dihydrotestosterone, testosterone, pregnenolone, 17-alpha-hydroxypregnenolone; 5-alpha-androstane-3b,17b-diol; 2-hydroxyestradiol, and 4-hydroxyestradiol.

According to the present invention, the preparation of a device with a molecular imprinted polymer having sites of selective recognition of secosteroid molecules includes the steps of (a) contacting TEOS monomers with one or more template molecules with similar or identical structure to one or more secosteroids of interest, (b) applying the mixture of (a) as a thin film on a support and (c) extracting the template molecules from the resulting polymer to form recognition sites of said secosteroid molecules.

The polymerization can be carried out in the absence of a catalyst by subjecting the reaction mixture to appropriate polymerization parameters, such as light, heat and pressure. If necessary, a catalyst can be used according to the nature of the polymerization. It can also be desirable to include a cross-linking agent in the reaction mixture. The polymerization can be carried out in the presence of a solvent and/or another porogenic agent. Polymerization reactions and conditions are known to those skilled in the art.

Thin films of the invention can be produced by any conventional method. However, the ability to control the thickness and formulate the films in an environment typical of printed Circuit production is an important feature of film production. Thus, in particular embodiments, the instant films are produced by phase inversion-spin coating onto a suitable support. The wet phase inversion procedure (Wang, et al. (1997) Langmuir 13:5396; Shibata, et al. (1999) J. Appl. Poly. Sci. 75:1546; Trotta, et al. (2002) J. Membr. Sci. 201:77) for preparation of MIPs involves a polymerized starting material that is dissolved with the template in a theta solvent. A template-host network is allowed to form in solution and precipitated by immersion in a non-solvent. Originally developed to produce MIP membranes, this procedure has been adapted to the production of thin, 300 nm to 5 μm, films via spin coating (Crabb, et al. (2002) J. Appl. Polym. Sci. 86:3611; Richter, et al. (2006) J. Appl. Polym. Sci. 101:2919; Campbell, et al. (2009) Surface and Interface Analysis 41:347) and hydrogen bond interactions between the template and host polymer.

The support of the instant molecular imprinted film can be a rigid or flexible material, which may be conducting, semiconducting or dielectric. The substrate can be a monolithic structure, or a multilayer or other composite structure having constituents of different properties and compositions. Suitable support materials include quartz, glass, alumina, mica, silicon, III-V semiconductor compounds, and other suitable materials. Optionally, additional electronic elements may be integrated into the support for various purposes, such as thermistors, integrated circuit elements or other elements.

The template molecules can be extracted by any known method that enables the template molecule to be removed without destroying the imprinted polymer. The extraction can be performed using a solvent containing or not a competitor agent or require chemical cleavage, such as hydrolysis, acid hydrolysis, alkaline hydrolysis, hydrogenation, reduction or oxidation.

As demonstrated herein, the device of the present invention was found to readily adsorb the secosteroid Vitamin D₃. Accordingly, the instant device finds application in a method for detecting secosteroids and metabolites thereof in a test sample, e.g., in the diagnosis of Vitamin D deficiency or Vitamin D toxicity. Test samples appropriate for the method described herein include any biological fluid, cell, tissue, or fraction thereof, containing or suspected of containing a secosteroid or metabolite thereof. A test sample can be, for example, a specimen obtained from an individual (e.g., a mammal such as a human) or can be derived from such an individual. For example, a test sample can be a biological fluid specimen such as blood, serum, plasma, urine, lachrymal fluid, and saliva. A test sample can be further fractionated, if desired, to a fraction containing particular cell types. For example, a blood sample can be fractionated into serum or into fractions containing particular types of blood cells. If desired, a test sample can be a combination of samples from an individual such as a combination of a tissue and fluid sample, and the like. Methods for obtaining test samples that preserve the activity or integrity of molecules in the sample are well known to those skilled in the art. Such methods include the use of appropriate buffers and/or inhibitors, including nuclease, protease and phosphatase inhibitors, which preserve or minimize changes in the molecules in the sample. Such inhibitors include, for example, chelators such as ethylenediamne tetraacetic acid (EDTA), ethylene glycol bis(Paminoethyl ether)N,N,N1,N1-tetraacetic acid (EGTA), protease inhibitors such as phenylmethylsulfonyl fluoride (PMSF), aprotinin, leupeptin, antipain and the like, and phosphatase inhibitors such as phosphate, sodium fluoride, vanadate and the like.

The amount of secosteroid in a test sample can be determined by measuring the absolute or relative amount (e.g., as compared to a calibration curve) of the secosteroid bound to the MIP film. To detect binding between a molecular imprinted film and a template molecule, the instant device can further include one or more sensors. Any suitable electrical property may provide the basis for sensor sensitivity, for example, electrical resistance, electrical conductance, current, voltage, capacitance, transistor on current, transistor off current, and/or transistor threshold voltage. In the alternative, or in addition, sensitivity may be based on a measurements including a combination of properties, relationships between different properties, or the variation of one or more properties over time. In some embodiments of the instant invention, the sensor is a capacitive sensor, a conductive sensor or a combination thereof. Depending on the type of sensor, the sensor can be a separate element of the device or integrated with the molecular imprinted film.

Information collected according to the methods provided herein can be used to assess the health state of a mammal (e.g., a human patient), such as presence or absence of a disorder (e.g., vitamin D deficiency, a steroid imbalance) or to evaluate the risk of developing such a disorder based on an excess or deficiency of a secosteroid. For example, vitamin D metabolites are involved in many important biological processes. Thus, abnormal increases or decreases in their levels can alter biological functions within an organism. A deficiency in vitamin D metabolites is associated with several human diseases including rickets, osteomalacia, osteoporosis, hypocalcemia, and hyperparathyroidism. An excess of vitamin D metabolites can be associated with hypercalcemia. Levels of circulating vitamin D metabolites have been associated with the potential for developing certain types of cancer. Higher serum 25-hydroxyvitamin D₃ levels have been associated with a decreased colorectal adenoma risk. See Peters, et al. (2004) Cancer Epidemiology Biomarkers & Prevention 13:546-552. In some cases, the method provided herein can be used to determine whether or not a mammal (e.g., a human individual) has a health state associated with altered levels of a secosteroid or metabolite thereof. In particular, levels of a secosteroid, or metabolite thereof, can serve as biochemical indicators of a disorder, regardless of whether physiologic or behavioral symptoms of the disorder are manifest in the individual.

The method described herein is also useful for determining therapeutic efficacy of a particular treatment. When a treatment is selected and treatment starts, the individual can be monitored periodically by collecting biological samples at two or more intervals, measuring the expression level of a secosteroid, or metabolite thereof, corresponding to a given time interval pre- and post-treatment, and comparing expression levels over time. On the basis of any trends observed with respect to increasing, decreasing, or stabilizing levels, a clinician or other health-care professional may choose to continue treatment as is, to discontinue treatment, or to adjust the treatment plan with the goal of seeing improvement over time. In some cases, the individual can be offered additional or alternative therapeutic options. In some cases, changes in the level of a particular a secosteroid or metabolite can indicate compliance or non-compliance with a particular treatment plan. For example, lower than expected serum levels of 25-hydroxyvitamin D₂ can indicate the individual's non-compliance with or poor response to a therapeutic regimen of vitamin D supplements. Therefore, the method and device provided herein are applicable to screening, diagnosis, prognosis, monitoring therapy and compliance, and any other application in which determining the amount of a secosteroid or metabolite thereof, such as a vitamin D metabolite, is useful.

Also provided herein are kits useful for determining the amount of a secosteroid, or metabolite thereof, in a test sample, as is described herein. Typically, a kit includes a device of the invention and a standard, calibration curve and/or information providing absolute amounts of a secosteroid, or metabolite thereof, indicative or normal or aberrant levels.

The invention is described in greater detail by the following non-limiting examples.

### EXAMPLE 1

### Preparation of MIP With TEOS

The host polymer solution was composed of 4 mL of TEOS, 1 mL of 0.1 M hydrochloric acid, and 1.2 mL of ethanol. The MIP was made by adding 500 mg of pure cholecalciferol (Vitamin D₃) into the solution. At the same time, a control solution was made following the same formula without the cholecalciferol. All solutions were allowed to mix for 24 hours, allowing ample time for the sol to non-covalently combine with the template.

To prepare a thin film, a glass substrate was rinsed with acetone, and then placed into the spin coater for 30 seconds at 5000 rpm; this provided a clean surface for the application of the host polymer solution. Subsequently, 0.12 mL of host polymer solution was applied to the surface of the glass substrate of size 25 mm×25 mm. It was important to manually cover the entire surface, or the spin coating process would leave uncoated patches. The substrate was then made to spin for 30 seconds at 5000 rpm.

During the mixing process the hydroxyl group of the Vitamin D₃ interacted via hydrogen bonding with the sol, so that the polar end of the Vitamin D₃ with the hydroxyl was embedded into the polymer and lightly held in place by the hydrogen bond. During the spin coating process, the gel formed around the template, creating a cavity in the shape of the template molecule. This template molecule could be removed by a solvent, which did not affect the gel polymer, and left a hole that accepted only molecules in the shape of the original template molecule. By means of another solution made with a solvent and the template molecule, the MIP could adsorb a certain measurable amount of template from the solution. If the template is in the presence of other molecules, only the template would be adsorbed.

To estimate the binding constant of the MIP, three different time trials with different solvents were conducted. These solvents were ethanol, methanol, and chloroform, and one gram of Vitamin D₃ was added per liter of said solvents. Chloroform readily reinserted small amounts of Vitamin D₃ into the polymer, whereas ethanol and methanol required water to be added as a co-solvent. One liter of water was added per one liter of ethanol and methanol. Five milliliters of each solution was added to two bottles with closeable lids. Each substrate was placed into the bottle for one hour, and then was scanned with a Fourier Transform InfraRed (FT/IR) Spectrometer. After each template was removed from the solution, a small amount was left on the surface. The solvent was evaporated and blotches of high concentration of Vitamin D₃ remained. Special care was taken to avoid any white blotches and multiple scans were undertaken to make sure that the given results were consistent. After a few repetitions, however, the entire substrate was covered with deposits. Washing the substrate off with water, after removing it from the solvent, solved the problem of the blotches, but the Vitamin D₃ was also removed by the process, and thus creating irreproducible results.

### EXAMPLE 2

### FT/IR Spectroscopy Analysis of MIP

To measure Vitamin D₃ in the system, the substrate was placed in the FT/IR Spectrometer. Once the presence of Vitamin D₃ was measured, the substrate was placed into a 10 mL ethanol bath for 1 hour. After the hour had concluded, the substrates were removed from the ethanol and allowed to dry. The substrates were then scanned again. The polymer not only had the template molecule embedded on the surface, but it was also found within the polymer. Scanning the substrates before and after the ethanol baths gave a cognitive number to the amount of Vitamin D₃ on the surface of the template, as well as how much remained in the polymer.

### EXAMPLE 3

### Scanning Electron Microscope (SEM) Analysis of MIP

The SEM granted deeper insight to the morphological differences between the different stages of the MIP process. TEOS was converted essentially into glass, and the morphology of the initial, as produced, MIP showed itself to be flat, have large cracks, and have darker colored circles. The morphology greatly changed after the ethanol baths. Though the surface retained its flat characteristics, it now had more cracks that were larger in size. The contents of the dark circles from the “as produced” substrate had been dissolved by the ethanol bath. It was discovered that the size of the bubbles were related to the speed of the spin coating process, amount of Vitamin D₃ added to the initial solution, and the amount of water added to the initial solution. Vitamin D₃ solution was applied to a substrate and was allowed to dry without the aid of the spin-coater. This produced a visible morphology that was similar to the surface of the SEM scans. One large bubble was scraped off the substrate, dissolved in 70 pL ethanol, and scanned in a UV spectrometer. It produced the spectra corresponding to Vitamin D₃. The dark circles were actually places where Vitamin D₃ had aggregated. The SEM images of the reinsertion of Vitamin D₃ showed that, to some degree, the template was reinserting itself into the cavities.

### EXAMPLE 4

### Substrate Recognition of MIP

To cognitively determine the amount of Vitamin D₃ in the substrate, the area function of the Jasco computer program was used. Because the peaks at 2948 cm^{p31 1} and 2870 cm¹ were the largest, the area under these peaks was used to determine the adsorption of Vitamin D₃ per unit time. Though the initial area of every substrate could vary greatly, the average initial area of these Vitamin D₃ substrates was 0.229, and the average area of the control substrates was 0.0281. The amount of Vitamin D₃ inside the polymer after the ethanol bath also varied, the average area was 0.0499 after the ethanol bath. These time trials were performed at one hour intervals, so that it would be possible to find the maximum adsorption. Every time a substrate was removed from its solution, the solution that remained on the surface evaporated to leave. Vitamin D₃ on the surface. The first part of this test was performed once per hour until the substrates were covered completely, which occurred at 180 minutes. The same substrates were placed into another ethanol bath, in order to completely remove the Vitamin D₃. This was carried out so that each set of substrates has a different initial concentration of Vitamin D₃. The new, clean substrates were placed into their corresponding solution for three hours, and then measured. Then the substrates were allowed to soak for one more hour, because the subsequent hour had the template coating to the surface.

Based upon this analysis, TEOS was found to provide a suitable sensor system for Vitamin D₃. From the experiments herein, it was found that a methanol solution reached its maximum adsorption after one hour, ethanol reached its maximum adsorption after two hours, and chloroform reached its maximum adsorption after three hours (Table 1). The maximum adsorption of each solvent was different, but this was due to the inherit differences of each MIP. Methanol had the highest rate of adsorption of the three solvents, and chloroform had the lowest.

TABLE 1

| Time | Average | | | |
|---|---|---|---|---|
| (Minutes) | Control | Ethanol | Methanol | Chloroform |
| 0 | 0.0246 | 0.0431 | 0.0652 | 0.0413 |
| 60 | 0.0198 | 0.0979 | 0.1300 | 0.0700 |
| 120 | 0.0330 | 0.1639 | 0.1313 | 0.0974 |
| 180 | 0.0391 | 0.1504 | 0.1225 | 0.1405 |
| 240 | 0.0354 | 0.1675 | 0.1349 | 0.1430 |

What is claimed is:

1. A device for detecting a secosteroid, or metabolite thereof, comprising
(a) a support; and
(b) a molecular imprinted tetraethoxysilane polymer film having sites of selective recognition for a secosteroid, or metabolite thereof.

2. The device of claim 1, wherein the film is produced by phase inversion-spin coating on the support.

3. A method for detecting a secosteroid, or metabolite thereof, in a test sample comprising:
(a) exposing the device of claim 1 to a test sample, and
(b) sensing binding of a secosteroid, or metabolite thereof, in the test sample to the device, thereby detecting the secosteroid, or metabolite thereof, in the test sample.

4. A kit comprising the device of claim 1.
